(19) United States
(12) Patent Application Publication
(10) Pub. No.: US 2011/0263922 A1
(43) Pub. Date: Oct. 27, 2011

(54) METHOD, DEVICE AND APPARATUS SYSTEM FOR PROSTATE CANCER THERAPY

(76) Inventors: Susanne Dornberger, Erlangen (DE); Jens Fehre, Hausen (DE); Rale Nanke, Neunkirchen am Brand (DE)

(21) Appl. No.: 13/086,623

(22) Filed: Apr. 14, 2011

(30) Foreign Application Priority Data
Apr. 22, 2010 (DE) 102010028105.0

(51) Int. Cl. A61N 5/00; A61B 5/05; A61B 5/00; A61B 18/02; A61B 18/18; A61B 10/00; A61B 18/20

(52) U.S. Cl. 600/3; 600/562; 600/476; 606/13; 606/21; 606/41; 600/300

(57) ABSTRACT

In a method, a device and an apparatus system for therapy of prostate cancer, in the course of a therapy session the prostate of a patient is subjected at different locations to a diagnostic examination with regard to the presence of a tumor, and in the case of a positive diagnosis the prostate is therapeutically treated at the corresponding location during the therapy session. The device has a hollow needle, a biopsy needle carrying an extraction element at its forward end that serves to extract a tissue sample, and a therapy element serving to therapeutically act at a location of the prostate. The biopsy needle or the optical waveguide and the therapy element are designed so that they can be inserted into the hollow needle. The apparatus system includes a diagnostic unit and a therapy unit for focal therapy treatment of the prostate, the diagnostic unit including a device for histological assessment of prostate tissue.

### BACKGROUND OF THE INVENTION

1. Field of the Invention

The present invention concerns a method and a device and an apparatus system for prostate cancer therapy.

2. Description of the Prior Art

According to the modern prior art, two standard methods can be selected to treat the prostate tumor (PCa), namely prostatectomy (the radical removal of the prostate) or what is known as active surveillance (“watchful waiting”).

The diagnosis normally takes place by palpation and/or by determining the PSA value in the blood of the patient. Since neither method allows a definitive conclusion as to whether PCa is present or not, in the event of suspicion a punch biopsy of the prostate is conducted. Under ultrasound monitoring (TRUS), the urologist punctures the prostate with a biopsy needle according to a specific puncture pattern, since PCa is normally not detectable in ultrasound. For example, 12 biopsy specimens are extracted—named according to their approximate extraction point (left/right; apex/middle/base; peripheral zone/transitional zone)—and sent to a pathologist for histological assessment. The result is provided to the urologist after a few days or weeks. Due to the blind extraction, often all biopsy specimens are negative, such that often a repeat biopsy is required for further clarification. So that the prostate can be punctured at different points in the repeat biopsy, knowledge of the extraction points of the previous biopsy is required. Items known as biopsy grids are used for this purpose, for example. These are plate-shaped structures with a predetermined hole pattern that are affixed to the biopsy apparatus, for example. Given a repeat biopsy, the biopsy needle is directed through other holes of the grid.

Using the histology data that is possibly present after multiple biopsies, the urologist in consultation with the patient makes a decision about a therapy, most often for a complete removal of the prostate with the known consequences of incontinence and impotence. The procedure is conducted either as an open operation or as a RALP (Robot Assisted Laparoscopic Prostatectomy). In addition to this, other forms of therapy exist, for instance hormone therapy and different forms of local therapy such as radiation therapy, brachytherapy and ablative methods (for instance cryotherapy and laser therapy). In local therapy, the entire prostate is normally subjected to an ablation. The reason for this is the poor ability to depict PCa with conventional imaging methods, and therefore the lack of knowledge about the precise position and propagation of PCa in the prostate. At best, given the presence of one or two positive findings with low Gleason score in one prostate half, a hemi-ablation is considered. Therefore, generally the treatment has previously entailed a massive damage to healthy prostate tissue, with the known side effects. With newer biopsy apparatuses, the extraction points of the biopsy specimens can be stored. Nevertheless, these apparatuses do not allow a targeted therapy of only the tumor tissue. This is due to the fact that during the course of the therapy the prostate has varied in size, shape or position relative to the point in time of the biopsy. The bladder presses on the prostate and alters its shape and/or position more or less depending on the fill level. Such a variation can also be caused by a rectally inserted apparatus, for instance an ultrasound probe. Therefore, it is also the case that an exact focusing (localization) of the therapy on a tumor cannot take place with the cited biopsy apparatuses. This is particularly true for PCa in the early stage, which already can barely be detected with conventional imaging methods due to its low volume.

### SUMMARY OF THE INVENTION

An object of the invention is to provide a method, a device and an apparatus system that allow focused therapy of a prostate tumor in which healthy prostate tissue is damaged as little as possible.

This object is achieved by a method according to the invention, in which a treatment of the tumor occurs only as necessary even during the therapy session, with factors affecting position and size of the prostate—such as bladder fill level or a different position of a newly rectally inserted ultrasound switching head—being excluded, or at least having only an insignificant effect.

In the method in accordance with the present invention, during the course of a treatment session in the context of prostate cancer therapy, the prostate of a patient is subjected to a diagnostic examination at different locations to identify the presence or absence of a tumor at the respectively different locations. In the case of a positive diagnosis (i.e., a diagnosis that a tumor is present at a respective location of the prostate, only the location in the prostate at which the positive diagnosis occurred is therapeutically treated during the treatment session.

An extracted tissue sample can be conducted during a single treatment session with the use of a diagnostic unit that embodies a device for histological assessment, such that the finding is present even during the treatment session. A PCa therefore can be treated immediately, for instance with the use of laser beams. In contrast, given a conventional procedure in which the tissue samples are examined by an external laboratory the finding is only available after a few days or weeks.

The histological assessment can be based on a known biopsy, meaning that tissue samples are extracted at specific points of the prostate, and these samples are subjected to a histological assessment immediately on site—i.e. while a patient is located on the patient table within the scope of a treatment session. In order to ensure the ability for a later re-location of a specific, positively assessed point of the prostate, the extraction points are marked or acquired and stored with the aid of an imaging method. For example, marking with a biopsy grid is possible.

A spatially precise acquisition and storage of extraction points is possible with the aid of an imaging method and a software to store the respective spatial positions of the extraction points. For example, such a system is the system under the name “TargetScan”, distributed by the company “Envisioneering Medical Technologies”, St. Louis, Mo. 63114. In a preferred method variant, the biopsy is implemented with the use of a device having a hollow needle and a biopsy needle that is movable and can be directed within the inner chamber of said hollow needle. After the tissue extraction, the hollow needle is left with unchanged position in the prostate and is only removed if a negative finding results. In the case of a positive diagnosis, a therapy—for example a laser ablation of the affected location—takes place via the internal chamber of the hollow needle. Since its position is unchanged relative to the point in time of the tissue extraction, a pinpoint therapy is possible. A storage of the spatial coordinates of the extraction point with the assistance of an imaging method and a storage medium is not required for this. All methods that can be implemented with the aid of a therapy element which can be introduced into the internal chamber of the hollow needle (thus rod-shaped, wire-shaped or fibrous elements) are thus suitable for the therapy. A device suitable for these method variants comprises a hollow needle; a biopsy needle bearing an extraction element at its forward end (the end serving for the extraction of a tissue sample); and a therapy element that, for example, is an optical waveguide with which a laser ablation can be conducted. However, other therapy forms such as cryotherapy, brachytherapy, PDT, electroporation and the administration of chemotherapeutics can also be considered.

In a preferred embodiment, an in vivo diagnosis in which a point of the prostate is charged via an optical waveguide with an electromagnetic radiation and the fluorescence response of the tissue is detected. Such a method is described in European Patent Application Nr. EP 09006583.0, the contents of which are incorporated herein by reference. In this method, living, tumor-afflicted tissue is detected in that an electromagnetic radiation is emitted towards this and the decay response of an eigenfluorescence intensity of the tissue (which eigenfluorescence intensity is excited by the electromagnetic radiation) is acquired with temporal and spectral resolution. A diagnosis result can already be obtained within a few seconds with such a method, which can also be implemented in a sample of living cells that is extracted via biopsy. For in vivo assessment it is advantageous that storage of diagnostically examined locations is not required.

In the event of a tissue extraction by means of a biopsy, to implement the method a device that comprises a hollow needle (the hollow needle embodying a biopsy needle bearing an extraction element at its forward end, the forward end serving for the tissue extraction, and a therapy element serving for therapeutic action at a point of prostate) instead of a conventional biopsy device. The biopsy needle and the therapy element—for example an optical waveguide—are designed so that they can be introduced into the hollow needle, advantageously singularly. However, the hollow needle could also be designed so that the biopsy needle and optical waveguide can be accommodated simultaneously. In addition to an optical waveguide with which a laser ablation can be conducted, a therapy element can also be considered with which the aforementioned therapy methods (such as cryotherapy etc.) can be applied.

In the case of an optical waveguide as a therapy element, it is advantageous that this is not switched out after the diagnosis, but rather that only therapeutic active laser light must be fed in. The optical waveguide is thus usable for diagnosis and therapy. For the case that a different therapy form such as laser ablation should be used, the device is designed similar to as is described further above, thus comprises a hollow needle, an optical waveguide for diagnostic purposes and a therapy element. The hollow needle is designed so that it can advantageously accommodate only one of the cited parts. In the in vivo method it is the case that the hollow needle containing the optical waveguide serving for diagnosis remains at the respective point after implementation of the diagnostic with unchanged position and alignment. A therapy taking place via the inner chamber of the hollow needle after the diagnostic can therefore be directed with pinpoint accuracy towards the tumor, and possibly its environment.

In the in vivo variant, the fluorescence of a fluorescence marker (known from DE 10 2007 028 659 A1, for example) can also be utilized in addition to the eigenfluorescence of the examined tissue (as described in Patent Application Nr. EP 09006583.0). Such a fluorescence marker is formed from a fluorescing molecule and a ligand, wherein the latter specifically binds to tumor tissue of the prostate. The fluorescence marker (supplied via the bloodstream, for example) accumulates [enriches] in tumor tissue, such that this is visible due to the fluorescence caused by exposure with light. For example, for detection of the fluorescence a fiberglass bundle can be used here and also in the diagnostic method known from the Patent Application Nr. EP 09006583.0. Naturally, the diagnosis based on fluorescence can also be applied extracorporeally given a sample extracted from the prostate.

An apparatus system suitable to implement a method of the type described above has a diagnostic unit and a therapy unit for focal therapy treatment of the prostate, wherein the diagnostic unit includes a device to histologically assess prostate tissue. With this basic configuration of medical apparatuses, for the aforementioned reasons a focal therapy can be implemented with pinpoint accuracy within the scope of a treatment session. It is advantageous for the apparatus system to also include an imaging device.

### BRIEF DESCRIPTION OF THE DRAWINGS

FIG. 1 schematically illustrates an apparatus system for diagnosis and focal therapy of a prostate tumor in accordance with the invention.

FIG. 2 is a significantly schematic section through a prostate, illustrating marking of extraction points in the image that are predetermined by a biopsy grid.

FIG. 3 illustrates biopsy of the prostate with the use of a conventional biopsy device.

FIG. 4 illustrates therapy of a PCa using laser light.

FIG. 5 illustrates brachytherapy of a PCa.

FIG. 6 illustrates therapy of a PCa by injection of a chemotherapy drug.

FIG. 7 illustrates electroporation of a PCa.

### DESCRIPTION OF THE PREFERRED EMBODIMENTS

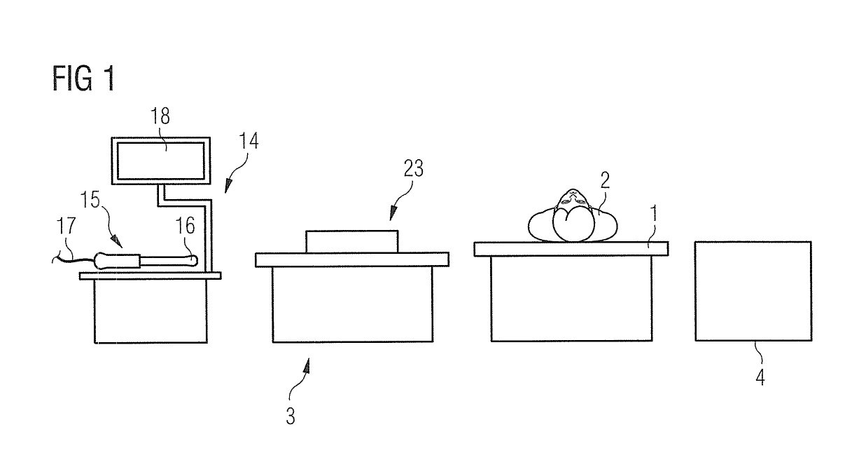

A workstation with an apparatus system to implement the method illustrated above is shown in a significantly schematic manner in FIG. 1. In addition to a patient table 1 to support a patient 2 (in particular in a horizontal position), the workstation is equipped with an apparatus system for diagnosis and therapy of a PCa of the prostate. The apparatus system has a diagnostic unit 3 and a therapy unit 4. In principle, the diagnostic unit 3 can comprise arbitrary medical apparatuses insofar as the sought goal (namely to locate a locally limited tumor in the prostate) can be achieved with these. For this purpose, in each case a device for histological assessment of prostate tissue is required. As is typical in previous methods, the tissue of the prostate that is to be extracted can be extracted with the aid of a biopsy. In this case the apparatus system comprises a biopsy device 6 that is shown in sections in FIG. 3. For example, it comprises a hollow needle 7 connected with a handle (not shown), in the inner chamber 8 of which hollow needle 7 a biopsy needle 9 is directed. For instance, this bears an extraction element 10 fashioned as a point, which extraction element 10 is provided with a barbed hook (not shown). If, starting from the position shown in FIG. 3, the biopsy needle 9 is drawn out of the prostate 12, tissue remains suspended on the barbed hook of the extraction element 10.

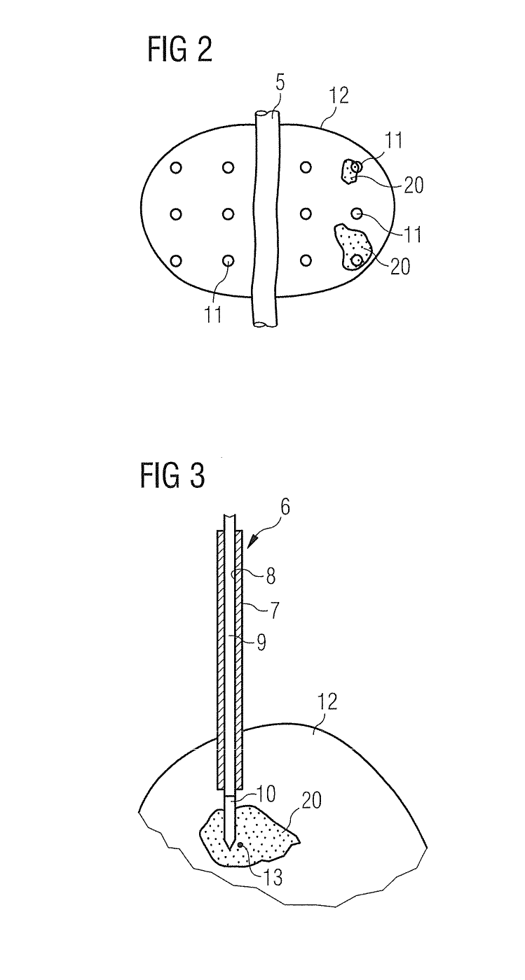

In the course of a biopsy, samples are extracted at multiple different points of the prostate. In order to monitor the tissue extraction visually on the one hand and, if necessary, to store the extraction points 13 recorded in the image on the storage medium of a PC (not shown), a device is present for imaging 14. For example, this comprises an ultrasound apparatus 15 with a rectally insertable ultrasound head 16. The ultrasound apparatus 15 is connected via a data line 17 with a computer (not shown) that in turn reproduces the prepared image information on a display 18. In addition to TRUS (transrectal ultrasound), other imaging methods such as MR or MR-US fusion can also be used. Different MRI protocols (T2W, DWI, DCE and MRS, for example) can thereby be used to show and delimit the tumor. Novel ultrasound methods such as 3D-TRUS, CE-US and elastography, or even molecular (for example optical) imaging methods can also be used. In addition to a storage of the spatial coordinates of an extraction point 13 with the aid of an imaging method an a storage medium, the use of what are known as biopsy grids (not shown) is also known. These are plate-shaped structures with a grid of bores, for example. The individual bores are numbered, such that an extraction point is defined by the number of the bore via which the hollow needle 7 has been inserted. The circles 11 rendered in the image of the prostate 12 (reference character 5=urethra) in FIG. 2 designate the respective positions of the cited bores of the biopsy grid. A relatively small PCa 20 (that is possibly still found in a very early development stage), cannot be made visualizable. In order to reasonably cover the entire region of the prostate with the biopsy, multiple (for example twelve) tissue samples are extracted. If necessary, additional biopsies can be conducted in the surroundings of an extraction point in order to clarify the size of a PCa.

The diagnostic unit 3 comprises a device 23 for histological assessment of prostate tissue. Such devices are known as such and are used in laboratories (for example) to which tissue samples have previously been sent for assessment. Since, according to the invention, diagnosis and therapy are conducted during a single treatment session, such devices or methods which already allow a finding after a short period of time (for example in less than 2 minutes) are naturally advantageous. Such a method described in EP Application Nr. 09 006 583.0 was already mentioned above. It operates with the aid of electromagnetic radiation with which the living tissue is excited to an eigenfluorescence radiation. The intensity of the eigenfluorescence is detected with a detector with constant scan rate for at least one wavelength, the difference autocorrelation function C(t) of the intensity decay response is determined with the determined intensity measurement values. From this the fractal dimension D_{F} for the respective exposed tissue is then calculated, and the value of the fractal dimension is compared with a tumor-specific threshold. If the threshold is exceeded, the exposed tissue of the tissue sample can be classified as afflicted with tumors. Additional details with regard to this method are to be learned from the cited EP Patent Application. The supply of the magnetic radiation can take place with the device described above, which comprises a hollow needle 7 and an optical waveguide 24. This thus represents the assessment [finding] device 23 or is at least a part of this.

The therapy unit 4 includes at least one device suitable for PCa therapy. All prevalent devices and methods to treat PCa can be used. This can be a device with which a therapy can be implemented from the group of: radiation therapy; HIFU (high-intensity focused ultrasound); cryotherapy*; brachytherapy* (seed/HDR); PDT* (photodynamic therapy); laser ablation*; electroporation*; administration of a chemotherapy agent*. The methods marked with * are implemented with the aid of elongated—for example rod-shaped, wire-shaped or fibrous—therapy elements. If a device with a hollow needle 7 (as in a variant of the proposed method) is now used for biopsy or diagnosis, after the tissue extraction or, respectively, diagnosis the inner chamber 8 of the hollow needle 7 can be exchanged for a different therapy element suitable to implement the cited therapies. Since the position of the hollow needle 8 remains unchanged, a focal therapy can take place with pinpoint accuracy with one of the cited methods and be limited to the PCa, and possibly to a tissue area surrounding this. Regions of the prostate 12 that are not afflicted by PCa thereby remain intact, such that in many cases the side effects—such as impotence and incontinence—linked with a complete removal of the prostate or with a large-volume ablation of prostate tissue do not occur.

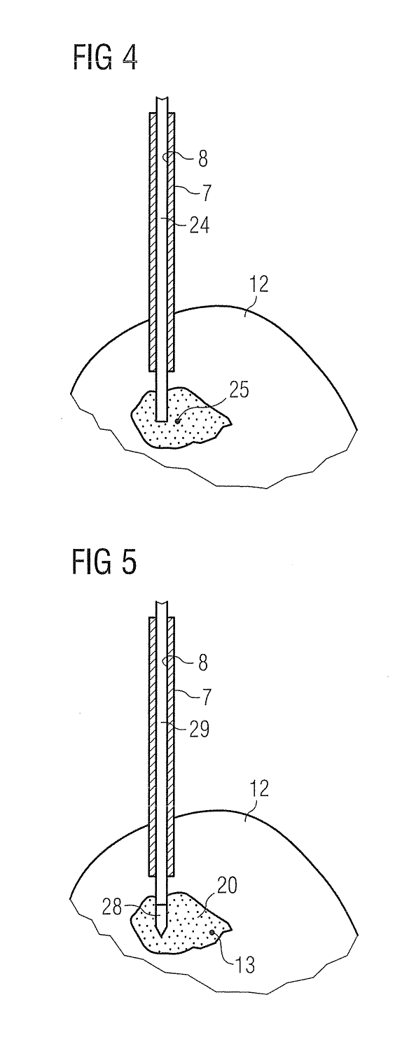

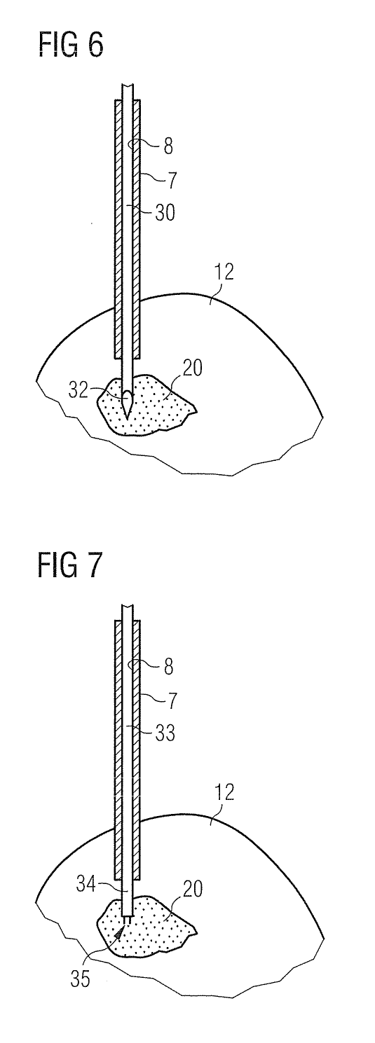

In conducting an in vivo diagnosis, via an optical waveguide 24 (FIG. 4) a point 25 in the prostate 12 is charged with electromagnetic radiation and the fluorescence response of the tissue is measured, as has already been described further above. In this method the eigenfluorescence of the PCa tissue is utilized. In a different procedure, a fluorescence marker is supplied to the prostate via the bloodstream, wherein this fluorescence marker is formed from a ligand binding specifically to PCa tissue and a fluorescing molecule bound to said ligand. The frequency of the electromagnetic radiation supplied via the optical waveguide 24 is thereby respectively selected so that it excites the molecule connected with the ligand to fluorescence. A fluorescence marker known from DE 10 2007 028 659 A1 is composed of a CEACAM1 antibody and the fluorescence dye NIR-1, for example. If a point 25 of the prostate 12 is detected as afflicted with a tumor, a therapy can be introduced immediately. In the sense of a pinpoint focal therapy it is advantageous for the optical waveguide 24 used for diagnosis to be introduced into the prostate with the aid of a hollow needle 26. In the case of a positive diagnostic, the internal chamber 27 of the hollow needle can be used as an access channel for the purpose of therapy at the location 25. The therapy forms already described further above—cryotherapy, brachytherapy, PDT, laser ablation, electroporation and administration of a chemotherapy agent—for whose implementation an elongated therapy element is required can thus be used. The therapy unit 4 in these cases comprises the respective therapy elements and the devices required for the respective therapy, for instance refrigeration units for cryotherapy, lasers for laser ablation and so forth. In the case of a laser ablation, the optical waveguide 24 used for diagnosis can also serve to supply the therapeutic laser light (FIG. 4). In the case of a brachytherapy, an LDR seed 28 (LDR=low dose rate) can be inserted into the prostate 12 with the use of a wire-shaped retaining tool 29 and the seed can be introduced into a PCa 20 (FIG. 5). The supply of a chemotherapy agent takes place via a cannula 30 inserted into the hollow needle 7, which cannula 30 is placed so that its exit opening 32 lies within the PCa 30 (FIG. 6).

An additional possibility of focal therapy is shown in FIG. 7. Here an electroporation element 33 which projects with an end region 34 (protruding from the hollow needle 7) into a PCa 20 is inserted into the inner chamber 8 of said hollow needle 7. An electrode pair 35 is present at the end region 34, for example on its facing side.

Although modifications and changes may be suggested by those skilled in the art, it is the intention of the inventor to embody within the patent warranted hereon all changes and modifications as reasonably and properly come within the scope of his contribution to the art.

We claim as our invention:

1. A method for diagnosis and therapy of prostate cancer, comprising the steps of:
implementing a treatment session of a patient for prostate cancer at a treatment apparatus at which the patient is located;
during said treatment session, while said patient is at said treatment apparatus, subjecting the prostate of the patient to a diagnostic examination at a plurality of different locations to determine, at each of said different locations, a presence or an absence of cancer tissue; and
in said treatment session, therapeutically treating only a location or locations for which said diagnostic examination produced a positive diagnosis indicating the presence of cancer tissue.

2. A method as claimed in claim 1 comprising implementing said diagnostic examination by extracting tissue samples in a biopsy procedure at each of said different locations while said patient is at said therapy apparatus, and implementing a diagnosis of each of said samples using a diagnostic unit located at a same site as said therapy apparatus, while said patient is at said therapy apparatus.

3. A method as claimed in claim 2 comprising generating location-identifying data for each of said different locations that allow subsequent identification of any of said locations at which a sample was extracted, using a location-identifying data generation procedure selected from the group consisting of marking said different locations and detecting said different locations with an imaging modality, and electronically storing said location-identifying data.

4. A method as claimed in claim 3 comprising implementing said biopsy procedure using a hollow needle and a biopsy needle that is moved in and directed by said hollow needle, and comprising marking each location by leaving said hollow needle in place in the prostate after extraction of said sample, while said sample is subjected to said diagnostic examination, and using said hollow needle for access to said location at said process to implement said therapeutic treatment at said location.

5. A method as claimed in claim 3 comprising selecting said imaging modality from the group consisting of TRUS, MR, and MR-US.

6. A method as claimed in claim 1 comprising implementing said diagnostic examination in vivo at a location of the prostate irradiated with electromagnetic radiation from an optical waveguide, and detecting a fluorescence response of the tissue irradiated with said electromagnetic radiation.

7. A method as claimed in claim 1 comprising applying a fluorescence marker to the prostate that binds specifically to cancer tissue in the prostate, and implementing said diagnostic examination in vivo by charging each location of the prostate, via an optical waveguide, with electromagnetic radiation, and detecting a fluorescence response of the fluorescence marker to said electromagnetic radiation.

8. A method as claimed in claim 7 comprising employing a fluorescent marker that is formed from a ligand that binds specifically to cancer tissue in the prostate, and a fluorescing dye associated with the ligand.

9. A method as claimed in claim 6 comprising introducing said optical waveguide into the prostate via a hollow needle inserted into the prostate, and leaving said hollow needle in the prostate after charging the location with said electromagnetic radiation, and while said diagnostic examination is implemented, and using said hollow needle to access said location for said therapeutic treatment if said diagnostic examination indicates a positive diagnosis at said location.

10. A method as claimed in claim 9 comprising implementing said therapeutic treatment by introducing a therapeutic instrument into the prostate through said hollow needle.

11. A method as claimed in claim 10 comprising implementing laser ablation with said instrument.

12. A device for contemporaneous diagnosis and therapy of prostate cancer of a patient, comprising:
a hollow needle configured for insertion into the prostate of the patient;
a biopsy needle carrying an extraction element at a leading end thereof operable to extract a tissue sample from the prostate;
a therapy element operable to therapeutically act at a location in the prostate; and
said biopsy needle having an inner lumen therein configured to allow insertion in succession of said biopsy needle followed by said therapy element to allow immediate diagnosis of said tissue sample with said hollow needle still in place in the prostate after removal of said biopsy needle therefrom, with subsequent introduction of said therapy element into said inner lumen of said hollow needle.

13. A device as claimed in claim 12 wherein said therapy element is an optical waveguide through which ablative radiation is administered.

14. A device as claimed in claim 12 wherein said therapy element is a cryoprobe.

15. A device as claimed in claim 12 wherein said therapy element is a retention tool.

16. A device as claimed in claim 12 wherein said therapy element is an LDR seed.

17. A device as claimed in claim 12 wherein said therapy element is an electrode pair to implement electroporation.

18. A device for contemporaneous implementation of diagnosis and therapy of prostate cancer of a patient, said device comprising:
a hollow needle configured for insertion into the prostate of a patient;
an optical waveguide that conducts electromagnetic radiation to a location in the prostate to irradiate the prostate with said electromagnetic radiation and that conducts, out of said waveguide, a fluorescence response of tissue in the prostate to said electromagnetic radiation;
a therapy element that administers cancer-treating therapy at a location of the prostate; and
said hollow needle having an inner lumen therein configured to receive, in succession, said optical waveguide and said therapy element to allow a diagnostic examination of said fluorescence response to be made while said hollow needle is still in place in said prostate, and for subsequent insertion of said therapy element into said inner lumen upon removal of said optical waveguide.

19. A device as claimed in claim 18 wherein said therapy element is an optical waveguide through which ablative radiation is administered.

20. A device as claimed in claim 18 wherein said therapy element is a cryoprobe.

21. A device as claimed in claim 18 wherein said therapy element is a retention tool.

22. A device as claimed in claim 18 wherein said therapy element is an LDR seed.

23. A device as claimed in claim 18 wherein said therapy element is an electrode pair to implement electroporation.

24. An apparatus for contemporaneous diagnosis and therapy of a prostate tumor in the prostate of a patient, comprising;
a patient bed;
a diagnostic unit configured to interact with the patient on the patient bed to obtain diagnostic information regarding prostate tissue in the prostate of the patient on the patient bed;
a therapy unit for administering localized therapy to the prostate of the patient on the patient bed; and
said diagnostic unit comprising a device configured for histological assessment of said prostate tissue, immediately upon obtaining said diagnostic information, while said patient remains located on said patient bed.

25. An apparatus as claimed in claim 24 comprising an imaging device that identifies a location of the prostate at which said diagnostic information was obtained, and for guiding operation of said therapy unit to administer said therapy treatment at the same location from which the diagnostic information was obtained.
